(19) United States
(12) Patent Application Publication
(10) Pub. No.: US 2023/0000409 A1
(43) Pub. Date: Jan. 5, 2023

(54) FATIGUE ESTIMATION SYSTEM, ESTIMATION DEVICE, AND FATIGUE ESTIMATION METHOD

(71) Applicant: Panasonic lntellectual Property Management Co., Ltd., Osaka (JP)

(72) Inventor: Kazuaki HASHIMOTO, Osaka (JP)

(21) Appl. No.: 17/781,689

(22) PCT Filed: Dec. 1, 2020
(86) PCT No.: PCT/JP2020/044731

§ 371 Date: Jun. 1, 2022

(30) Foreign Application Priority Data
Dec. 6, 2019 (JP) 2019-221235
May 28, 2020 (JP) 2020-092894

(51) Int. Cl. A61B 5/16; A61B 5/11; A61B 5/026; A61B 5/00; G06V 40/10

(52) U.S. Cl. CPC A61B 5/16; A61B 5/1116; A61B 5/026; A61B 5/6804; A61B 5/742; G06V 40/10

(57) ABSTRACT

A fatigue estimation system that includes: an information output device (imaging device) that outputs information (images) regarding the locations of the body parts of a subject; and an estimation device that estimates a posture of the subject based on the information output by the information output device, and estimates the fatigue level of the subject based on the posture estimated and the duration of the posture estimated.

### CROSS-REFERENCE OF RELATED APPLICATIONS

This application is the U.S. National Phase under 35 U.S.C. § 371 of International Patent Application No. PCT/JP2020/044731, filed on Dec. 1, 2020, which in turn claims the benefit of Japanese Patent Application No. 2019-221235, filed on Dec. 6, 2019, and Japanese Patent Application No. 2020-092894, filed on May 28, 2020, the entire disclosures of which Applications are incorporated by reference herein.

### TECHNICAL FIELD

The present disclosure relates to a fatigue estimation system for estimating the fatigue level of a subject, an estimation device for use in the fatigue estimation system, and a fatigue estimation method.

### BACKGROUND ART

In recent years, cases such that the accumulation of fatigue leads to poor health, injuries, accidents, etc. are found here and there. This has brought our attentions to technologies for preventing poor health, injuries, accidents, etc. by estimating the level of fatigue. For example, Patent Literature (PTL) 1 discloses, as a fatigue estimation system for estimating a fatigue level, a fatigue determination device that determines presence or absence of fatigue and the type of the fatigue, based on force measurement and bioelectrical impedance analysis.

### CITATION LIST

### Patent Literature

[PTL 1] Japanese Unexamined Patent Application Publication No. 2017-023311

### SUMMARY OF INVENTION

### Technical Problem

Unfortunately, with a conventional fatigue determination device as exemplified in the aforementioned PTL 1, the accuracy of an estimated fatigue level is not satisfactory in some cases. In view of this, the present disclosure provides, for instance, a fatigue estimation system that estimates a fatigue level with higher accuracy.

### Solution to Problem

A fatigue estimation system according to an aspect of the present disclosure includes: an information output device that outputs information regarding locations of body parts of a subject; and an estimation device that estimates a posture of the subject based on the information output by the information output device, and estimates a fatigue level of the subject based on the posture estimated and duration of the posture estimated.

An estimation device according to an aspect of the present disclosure includes: an obtainer that obtains information regarding locations of body parts of a subject; a posture estimator that estimates a posture of the subject based on the information obtained by the obtainer; and a fatigue estimator that estimates the fatigue level based on duration of the posture estimated by the posture estimator.

A fatigue estimation method according to an aspect of the present disclosure includes: obtaining information regarding locations of body parts of a subject; estimating a posture of the subject based on the information obtained in the obtaining; and estimating the fatigue level based on duration of the posture estimated in the estimating of the posture.

### Advantageous Effects of Invention

The fatigue estimation system according to an aspect of the present disclosure, for instance, can estimate fatigue with higher accuracy.

### BRIEF DESCRIPTION OF DRAWINGS

FIG. 1A is a first diagram for explaining the estimation of a fatigue level according to an embodiment.

FIG. 1B is a second diagram for explaining the estimation of a fatigue level according to the embodiment.

FIG. 1C is a third diagram for explaining the estimation of a fatigue level according to the embodiment.

FIG. 2 is a block diagram illustrating a functional configuration of a fatigue estimation system according to the embodiment.

FIG. 3 is a flowchart illustrating a fatigue estimation method according to the embodiment.

FIG. 4A is a diagram illustrating a subject who is static in posture A.

FIG. 4B is a diagram illustrating the subject who is static in posture B.

FIG. 5A is a first diagram for explaining the accumulation of an estimated fatigue level of the subject according to the embodiment.

FIG. 5B is a second diagram for explaining the accumulation of an estimated fatigue level of the subject according to the embodiment.

FIG. 6 is a first diagram illustrating an example of a display of an estimation result according to the embodiment.

FIG. 7 is a second diagram illustrating an example of a display of an estimation result according to the embodiment.

FIG. 8 is a diagram for explaining the estimation of a posture according to a variation of the embodiment.

### DESCRIPTION OF EMBODIMENTS

Hereinafter, embodiments of the present disclosure will be described with reference to the drawings. Note that each of the embodiments described below illustrates a generic or specific example. Moreover, numerical values, shapes, materials, elements, arrangement and connection of the elements, steps, an order of steps, etc. described in the following embodiments are mere examples and are not intended to limit the present disclosure. Among elements described in the following embodiments, those not recited in any one of the independent claims are described as optional elements.

Note that the drawings are schematic and are not necessarily accurate illustrations. Moreover, elements having substantially same configurations are assigned with like reference signs in the drawings, and duplicate description may be omitted or simplified.

### Embodiment

### Fatigue Estimation System

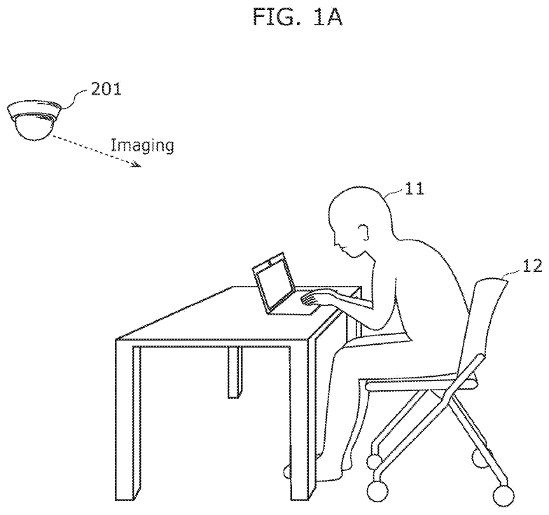

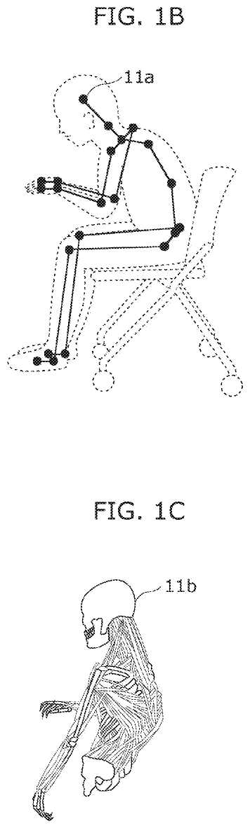

Hereinafter, an overall configuration of a fatigue estimation system according to an embodiment will be described. FIG. 1A is a first diagram for explaining the estimation of a fatigue level according to the embodiment. FIG. 1B is a second diagram for explaining the estimation of a fatigue level according to the embodiment. FIG. 1C is a third diagram for explaining the estimation of a fatigue level according to the embodiment.

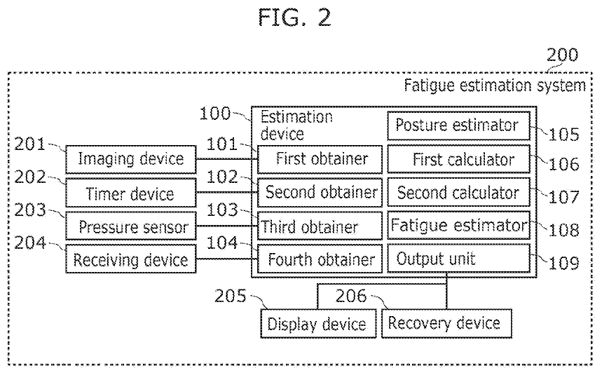

Fatigue estimation system 200 (see FIG. 2 to be described later) according to the present disclosure is a system for estimating the fatigue level of subject 11, using images that are output by imaging device 201 after the imaging of subject 11. The form of imaging device 201 is not limited to an example described in the embodiment, and may be a fixed camera provided on a wall or in the ceiling of a building, as illustrated in FIG. 1A, or a camera provided in a PC, a smartphone, a tablet device, etc. operated by subject 11, so long as it is a camera that captures images of subject 11 and outputs the images.

The subject here is in the posture of sitting on chair 12. In fatigue estimation system 200 according to the present disclosure, the fatigue level of subject 11 is estimated based on fatigue, among fatigue in subject 11, which is accumulated by subject 11 taking a static posture that is a fixed posture. In other words, fatigue, which is accumulated due to a load imposed on at least one of a muscle or a joint and a deteriorating blood flow (hereinafter also referred to as a decrease in a blood flow rate) which result from the fixed state of the posture, is estimated. Accordingly, subject 11 is in the static posture of sitting, lying, or standing for at least a certain period of time. The certain period of time is a minimum period such as several seconds or several tens of seconds in which fatigue can be estimated in fatigue estimation system 200. Such period of time is determined depending on the processing capacities of imaging device 201 and estimation device 100 (see FIG. 2 to be described later) included in fatigue estimation system 200.

Subject 11 who takes such a static posture is, for example, a desk worker in an office, a driver who maneuvers a moving body, a person who exercises for muscle training utilizing a load imposed by a static posture, a patient in a facility such as a hospital, a passenger or a crew in an airplane, etc.

Images captured and output by imaging device 201 is processed by estimation device 100, and the posture of subject 11 is estimated as illustrated in FIG. 1B. The estimated posture of subject 11 is output as, for example, a rigid link model. Specifically, skeletons indicated by straight lines are connected by joints indicated by black dots, and the posture of subject 11 can be reproduced based on positional relationships between the skeletons and the joints, each of which is a positional relationship between two skeletons connected by a single joint, as illustrated in FIG. 1B. The estimation of the posture is performed through image recognition, and the estimated posture is output as a rigid link model based on the positional relationships between the joints and the skeletons.

By applying the estimated rigid link model to a musculo-skeletal model as illustrated in FIG. 1C, an amount of load imposed on at least one of a muscle or a joint of each of body parts for keeping the positional relationships that are in accordance with the estimated posture is calculated as an estimated value for each of the body parts including muscles pulling skeletons and joints connecting the skeletons in such a manner that the positional relationships of the skeletons are changeable. The estimated value of the amount of load imposed on at least one of the muscle or the joint of the body part is accumulated more as duration in which the static posture continues gets longer. Accordingly, the level of fatigue caused by subject 11 keeping the static posture is calculated through computation using the estimated value of the amount of the load and the duration of the static posture. Note that “at least one of a muscle or a joint” is also expressed as “a muscle and/or a joint” in the following description.

In the present embodiment, it is possible to estimate a fatigue level based on the estimated value of the blood flow rate of subject 11 in addition to the estimated value of an amount of load imposed on a muscle and/or a joint of subject 11. The following description focuses on an example of estimating the fatigue level of subject 11 using the estimated values of an amount of load imposed on a muscle and an amount of load imposed on a joint, but it is also possible to estimate, with higher accuracy, the fatigue level of subject 11 by combining therewith the estimated value of the blood flow rate of subject 11. Furthermore, it is also possible to estimate the fatigue level of subject 11, using the estimated value of any one of an amount of load imposed on a muscle, an amount of load imposed on a joint, and the blood flow rate of subject 11.

In other words, in fatigue estimation system 200, after the posture of subject 11 is estimated, at least one of an amount of load imposed on a muscle, an amount of load imposed on a joint, or the blood flow rate of subject 11 is estimated based on the duration of the estimated posture. Fatigue estimation system 200 estimates the fatigue level of subject 11 based on the estimated value of at least one of an amount of load imposed on a muscle, an amount of load imposed on a joint, or the blood flow rate of subject 11. Hereinafter, for the sake of simplification, the estimated value of an amount of load may be simply expressed as an amount of load or an estimated value. When the estimated value includes the estimated value of a blood flow rate, “an amount of load” may be read as “a blood flow rate”, and “an increase in an amount of load” may be replaced with “a decrease in a blood flow rate” while “a decrease in an amount of load” may be replaced with “an increase in a blood flow rate”.

A blood flow rate is information for quantifying a blood flow that deteriorates due to subject 11 keeping a posture, as described above. The blood flow of subject 11 deteriorates more as the blood flow rate of subject 11 decreases, and a blood flow rate can be used as an index of fatigue caused by the deterioration of the blood flow. A blood flow rate may be obtained as an absolute numerical value at a measurement time point or as a relative change value between numerical values at two different time points. For example, how the blood flow of subject 11 is deteriorated can be estimated by the posture of subject 11 and relative numerical values indicating the blood flow rates at two time points of the start point and the end point of the posture. Alternatively, the blood flow rate of subject 11 may be estimated based simply on the posture of subject 11 and the duration of the posture since there is a correlated relation between (i) the posture of subject 11 and the duration of the posture, and (ii) the deterioration of the blood flow.

In the following description, at least one of an amount of load imposed on a muscle, an amount of load imposed on a joint, or the blood flow rate of subject 11 is estimated from the posture of subject 11, using the musculo-skeletal model, but besides the musculo-skeletal model, a method that uses measured data can be also applied as a method for estimating an amount of load imposed on a muscle, an amount of load imposed on a joint, and the blood flow rate of subject 11. In other words, the measured data is a database created by accumulating, in association with a posture, the measured values of an amount of load imposed on a muscle, an amount of load imposed on a joint, and the blood flow rate of subject 11 which are measured for each posture. With fatigue estimation system 200 in this case, by inputting the estimated posture of subject 11 into the database, it is possible to obtain, as output, the measured values of an amount of load imposed on a muscle, an amount of load imposed on a joint, and the blood flow rate of subject 11 for the posture that has been input.

Measured data may be created by using measured values obtained for each person in consideration of differences among subjects 11 or by optimizing, for each subject 11, big data obtained from a large number of unspecified subjects through statistical analysis or analysis processing such as machine learning.

Next, a functional configuration of fatigue estimation system 200 according to the present disclosure will be described with reference to FIG. 2. FIG. 2 is a block diagram illustrating a functional configuration of the fatigue estimation system according to the embodiment.

As illustrated in FIG. 2, fatigue estimation system 200 according to the present disclosure includes estimation device 100, imaging device 201, timer device 202, pressure sensor 203, receiving device 204, display device 205, and recovery device 206.

Estimation device 100 includes first obtainer 101, second obtainer 102, third obtainer 103, fourth obtainer 104, posture estimator 105, first calculator 106, second calculator 107, fatigue estimator 108, and output unit 109.

First obtainer 101 is a communication module that is connected to imaging device 201 and obtains, from imaging device 201, images in each of which subject 11 is captured. In other words, first obtainer 101 is an example of an obtainer, First obtainer 101 is connected to imaging device 201 by wires or wirelessly, and a method of communication performed via the connection is not specifically limited.

Second obtainer 102 is a communication module that is connected to timer device 202 and obtains a time from timer device 202. Second obtainer 102 is connected to timer device 202 by wires or wirelessly, and a method of communication performed via the connection is not specifically limited.

Third obtainer 103 is a communication module that is connected to pressure sensor 203 and obtains a pressure distribution from pressure sensor 203. Third obtainer 103 is connected to pressure sensor 203 by wires or wirelessly, and a method of communication performed via the connection is not specifically limited.

Fourth obtainer 104 is a communication module that is connected to receiving device 204 and obtains personal information from receiving device 204. Fourth obtainer 104 is connected to receiving device 204 by wires or wirelessly, and a method of communication performed via the connection is not specifically limited.

Posture estimator 105 is a processing unit implemented by a predetermined program being executed using a processor and memory. The posture of subject 11 is estimated through processing performed by posture estimator 105 based on images obtained by first obtainer 101 and a pressure distribution obtained by third obtainer 103.

First calculator 106 is a processing unit implemented by a predetermined program being executed using a processor and memory. An amount of load imposed on each of muscles and/or joints is calculated through processing performed by first calculator 106 based on the estimated posture of subject 11 and personal information obtained by fourth obtainer 104.

Second calculator 107 is a processing unit implemented by a predetermined program being executed using a processor and memory. A recovery amount of fatigue in each of muscles and/or joints is calculated through processing performed by second calculator 107 based on the amount of change in a change of the estimated posture of subject 11.

Fatigue estimator 108 is a processing unit implemented by a predetermined program being executed using a processor and memory. Fatigue estimator 108 estimates the fatigue level of subject 11 based on the duration of an estimated posture, using a posture estimated by posture estimator 105 and times obtained by second obtainer 102.

Output unit 109 is a communication module that is connected to display device 205 and recovery device 206 and that outputs contents that are based on the result of the estimation of a fatigue level performed by estimation device 100 to display device 205 and recovery device 206. Output unit 109 is connected to display device 205 or recovery device 206 by wires or wirelessly, and a method of communication performed via the connection is not specifically limited.

Imaging device 201 is a device that captures images of subject 11 and outputs the images, and is implemented by a camera, as described above. An existing camera such as a security camera or a fixed point camera may be used as imaging device 201 or a dedicated camera may be newly provided in a space in which fatigue estimation system 200 is installed. Such imaging device 201 is an example of an information output device that outputs images as information regarding the locations of the body parts of subject 11. Accordingly, the output information is images and each of the images includes the positional relationships of the body parts of subject 11 on an imaging sensor by which subject 11 is projected.

Timer device 202 is a device that measures a time, and is implemented by a clock. Timer device 202 can send a time to second obtainer 102 to which timer device 202 is connected. The time measured by timer device 202 here may be an absolute time or a relative elapsed time from a start point on a time axis. Timer device 202 may be implemented in any kind of form as long as it is possible to measure a time between two time points that are a time point at which the static state of subject 11 is detected and a time point at which the fatigue level of subject 11 is estimated (i.e., duration in which the static posture of subject 11 continues).

Pressure sensor 203 is a sensor having a detection face and measures pressure given to each of unit detection faces obtained by sectioning the detection face into one or more sections. Pressure sensor 203 thus measures pressure for each of the unit detection faces and outputs a pressure distribution on the detection face. Pressure sensor 203 is provided in such a manner that subject 11 is on the detection face.

Pressure sensor 203 is provided, for example, on the seat and the back rest of a chair on which subject 11 is seated. For example, a marker may be placed on the detection face of pressure sensor 203, and subject 11 may be guided to the detection face by a display showing a message such as “Please be seated on the marker”. By guiding subject 11 to the detection face of pressure sensor 203 provided on a portion of a floor, pressure sensor 203 may output a pressure distribution for subject 11 on the floor. Note that a pressure distribution is used with the view to enhance the accuracy of the estimation of a fatigue level, and therefore, fatigue estimation system 200 may be implemented without pressure sensor 203 when the satisfactory level of accuracy is ensured.

Receiving device 204 is a user interface that receives, as input, personal information of subject 11, and is implemented by an input device such as a touch panel or a keyboard. The personal information includes at least one of age, sex, height, weight, a muscle mass, a stress level, a proportion of fat in a body, or proficiency in performing exercise. The age of subject 11 may be a specific numerical value, or an age zone sectioned by ten years as in expressions such as teenage, twenties, and thirties, or an age zone defined by two sections with a predetermined age as a border as in an expression such as below 59 or over 60, or any other age zone.

The sex of subject 11 is an appropriate one selected out of male and female. Specific numerical values are received for the height and weight of subject 11. The compositional ratio of a muscle of subject 11 which is measured using, for instance, a body composition analyzer is received as the muscle mass of subject 11. The stress level of subject 11 is selected by subject 11 himself/herself from among, for instance, high, intermediate, and low as the subjective degree of stress felt by subject 11.

A proportion of fat in the body of subject 11 is a ratio of the weight of body fat percentage in the weight of subject 11, and is represented by, for example, percentage.

The proficiency of subject 11 in performing exercise may be quantified by scores attained when subject 11 performs exercise in a predetermined program, or by the conditions in which subject 11 performs exercise that subject 11 usually takes. In the former case, the proficiency is quantified by, for example, a time required for ten times of back extension, a time required for running 50 meters, or a flying distance achieved in making a long throw. In the latter case, the proficiency is quantified by, for example, how many days subject 11 performs exercise during a week or how many hours subject 11 performs exercise. Note that since personal information is used with the view to improve the accuracy of the estimation of a fatigue level, fatigue estimation system 200 may be implemented without receiving device 204 when the satisfactory level of accuracy is ensured.

Display device 205 is a device for displaying contents that are based on the estimation result of a fatigue level. Display device 205 displays an image indicating the contents that are based on the estimation result of a fatigue level, using a display panel such as a crystal liquid panel or an electroluminescent (EL) panel. The contents displayed by display device 205 will be described later. In the case of configuring fatigue estimation system 200 only to decrease the fatigue level of subject 11 using recovery device 206, only recovery device 206 needs to be included in fatigue estimation system 200 and display device 205 is not essential.

Recovery device 206 is a device for decreasing the fatigue level of subject 11 by promoting the blood circulation of subject 11. Specifically, recovery device 206 performs voltage application, pressurization, vibration, or heating, or changes the arrangement of the parts of chair 12 by means of a mechanism provided in chair 12, to actively change the posture of subject 11 who is seated on chair 12. Accordingly, recovery device 206 changes the condition of subject 11 defined by at least one of a load imposed on a muscle and a load imposed on a joint, and promotes the blood circulation of subject 11. By thus promoting the blood circulation, an influence made by the deterioration of blood flow caused by subject 11 taking a static posture is reduced, and the fatigue level of subject 11 is recovered also in terms of blood circulation. Recovery device 206 is wore on or in contact with an appropriate body part of subject 11 in advance in accordance with the configuration of recovery device 206.

Note that in the case of promoting the blood circulation of subject 11 by heating, since a whole space in which subject 11 is located is heated, there is no need for subject 11 to wear or be in contact with recovery device 206 on an appropriate part of the body. In the case of configuring fatigue estimation system 200 only to display the estimation result of a fatigue level to subject 11, only display device 205 needs to be included in fatigue estimation system 200 and recovery device 206 is not essential.

### Operation

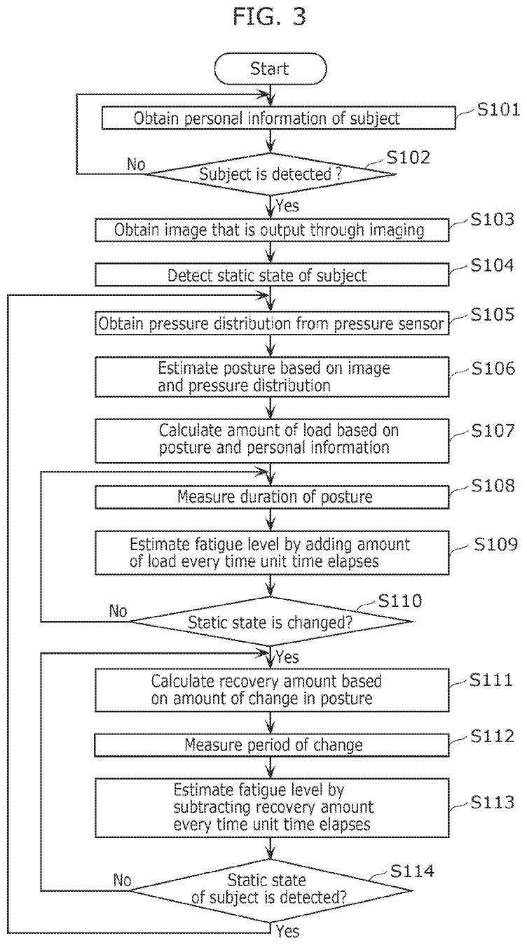

Next, the estimation of the fatigue level of subject 11 with the use of fatigue estimation system 200 according to the embodiment will be described with reference to FIG. 3 to FIG. 5B. FIG. 3 is a flowchart illustrating a fatigue estimation method according to the embodiment.

Fatigue estimation system 200 firstly obtains personal information of subject 11 (step S101). The obtainment of the person& information is performed by input of the personal information to receiving device 204 by, for instance, subject 11 or a manager who manages the fatigue level of subject 11. The personal information of subject 11 that has been input is stored in, for instance, a storage device not shown in the figure, and is then read out and used when the fatigue level of subject 11 is estimated.

Fatigue estimation system 200 detects subject 11, using imaging device 201 (step S102). The detection of subject 11 is performed by determining whether subject 11 appears in the angle of view of a camera that is imaging device 201. Note that subject 11 may be specific subject 11 or a person, among an unspecified number of persons, who appears in the angle of view of the camera, In the case where subject 11 is selected from the unspecified number of persons, the input of personal information may be omitted. In the case of detecting specific subject 11, a step of identifying subject 11 through image recognition or any other way is added.

The present embodiment describes an example in which the estimation of a fatigue level is performed by subject 11 himself/herself firstly inputting personal information, and then grasping a detection area that is set by imaging device 201, and entering the detection area. Accordingly, image recognition, for instance, is unnecessary and the fatigue level is estimated in consideration of the personal information.

In fatigue estimation system 200, when it is determined that subject 11 is not detected (No in step S102), step S102 is repeated until subject 11 is detected. When subject 11 is detected (Yes in step S102), images that are output by imaging device 201 are obtained by first obtainer 101 (step S103, an example of obtaining information regarding the locations of the body parts of a subject in the fatigue estimation method). When subject 11 is detected as being static (being in a static posture) in the obtained images (step S104), the posture of subject 11 is estimated by estimation device 100. Specifically, at first, third obtainer 103 obtains a distribution of pressure given to the detection face of pressure sensor 203 from pressure sensor 203 (step S105).

Posture estimator 105 estimates the posture of subject 11 based on the obtained images and pressure distribution (posture estimation step S106). When biased pressure is given to the detection face, for example, a pressure distribution is used to correct an estimated posture so that a bias is formed. Subsequently, first calculator 106 calculates an amount of load imposed on each of muscles and/or joints of subject 11 based on the result of the posture estimation, where the amount of load is corrected and calculated using the personal information obtained in advance (step S107). Note that the posture estimation of subject 11 and the calculation of the amount of the load are as described with reference to FIG. 1B and FIG. 1C, respectively, detailed description is omitted.

In the correction of the amount of load using personal information, the amount of load is decreased as the age of subject 11 is closer to the peak age of muscle development and is increased as the age of subject 11 gets away from the peak age. A value indicating such a peak may be based on the sex of subject 11. The amount of load may be decreased when the sex of subject 11 is male, and may be increased when the sex is female. Alternatively, the amount of load may be decreased as the height and weight of subject 11 indicate smaller values and may be increased as the height and weight indicate larger values.

Moreover, the amount of load may be decreased as the muscle mass of subject 11 has a larger compositional rate and may be increased as the muscle mass has a smaller compositional rate. The amount of load may be decreased as the stress level of subject 11 gets lower and may be increased as the stress level gets higher. Alternatively, the amount of load may be increased as the proportion of fat in the body of subject 11 gets higher and may be decreased as the proportion gets lower. Furthermore, the amount of load may be decreased as the proficiency of subject 11 in performing exercise gets higher and may be increased as the proficiency gets lower.

The duration of the static posture of subject 11 measured based on times obtained by second obtainer 102 (step S108).

Fatigue estimator 108 adds the calculated amount of load every time a unit time elapses in the duration of the static posture, and estimates the fatigue level of subject 11 at this time point (fatigue estimation step S109). The processes in step S108 and fatigue estimation step S109 continue until the static state of subject 11 changes.

Specifically, whether the static state changes or not is determined by determining whether the posture estimated by posture estimator 105 changes from a certain static posture (step S110).

When it is not determined that the static state changes (No in step S110), fatigue estimator 108 returns to step 5108, measures the duration of the static posture, proceeds to fatigue estimation step S109, and adds an amount of load, to accumulate the fatigue level of subject 11 as long as the static posture continues. In other words, by repeating the processes in step S108 and fatigue estimation step S109, fatigue estimator 108 estimates the fatigue level of subject 11 by using an increasing function that indicates the fatigue level with respect to the duration and has a slope corresponding to a calculated amount of load. Accordingly, the fatigue level of subject 11 increases per unit time as the calculated amount of load increases. Note that in such an accumulation of the fatigue level of subject 11, the fatigue level is reset (set to fatigue level 0) at a timing when a static posture starts which is a starting point on the time axis.

When it is determined that the static state changes (Yes in step S110), posture estimator 105 calculates the amount of the change in the posture from the original static posture to the current posture to which the static posture has changed. The amount of the change in the posture is calculated for each of muscles and/or joints, as is the case of the amount of load described above. When the posture thus changes, the load imposed on at least one of the muscle or the joint changes. When it comes to the blood flow rate of subject 11, the blood flow that has been deteriorating is temporarily improved and the fatigue level of subject 11 turns toward recovery. The fatigue level decreased by recovery pertains to the amount of the change in the posture. Accordingly, second calculator 107 calculates, based on the amount of the change in the posture, a recovery amount which is the degree of fatigue recovery (step S111).

Fatigue estimator 108 measures, based on times obtained by second obtainer 102, a period of change which is a time period during which the posture of subject 11 continues to change (step S112). A relationship between the recovery amount and the period of change is the same as that between the amount of load and the duration of the posture, and the recovery amount of subject 11 continues to be accumulated as long as the posture continues to change. In other words, at the timing when the posture of subject 11 thus changes, fatigue estimator 108 estimates the fatigue level of subject 11 by subtracting a recovery amount every time a unit time elapses (step S113).

Fatigue estimator 108 continues the processes in step S111, step S112, and step S113 until the posture of subject 11 is static. Specifically, fatigue estimator 108 determines whether the posture estimated by posture estimator 105 is a certain static posture (step S114). When the static state of subject 11 is not detected (No in step S114), fatigue estimator 108 returns to step S111, calculates a recovery amount, proceeds to step S112, measures a period of change, proceeds to step S113, and performs subtraction of the recovery amount, to continue this process so that the fatigue level of subject 11 recovers as long as the posture continues to change.

In other words, by repeating the processes in step S111, step S112, and step S113, fatigue estimator 108 estimates the fatigue level of subject 11 using a decreasing function that indicates the fatigue level with respect to the period of change and has a slope corresponding to the calculated recovery amount. Since the recovery amount of the fatigue level depends on the amount of the change in the posture, the fatigue level of subject 11 decreases per unit time as the amount of the change in the posture increases.

When the static state of subject 11 is detected (Yes in step S114), fatigue estimator 108 returns to step S105 and estimates again the posture and the fatigue level of subject 11 for a new static posture. Thus, in fatigue estimation system 200, since the fatigue level of subject 11 is calculated in consideration of the duration of a static posture based on images, it is possible to estimate the fatigue level of subject 11 with higher accuracy and a less amount of load imposed on subject 11.

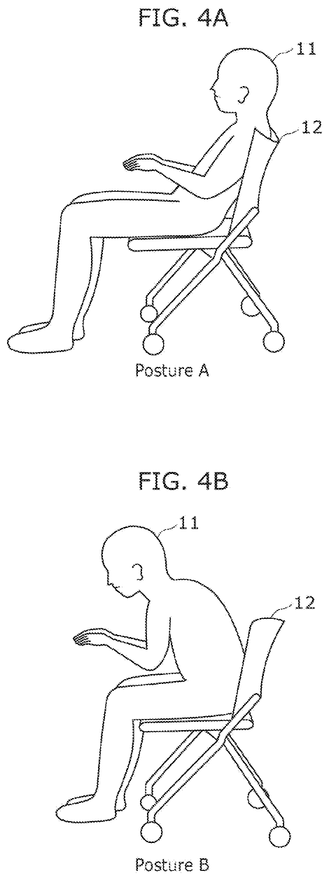

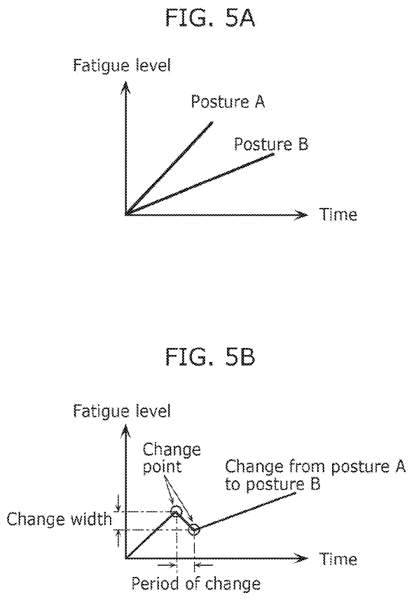

What has been described above will be described in detail with reference to FIG. 4A to FIG. 5B. FIG. 4A is a diagram illustrating a subject who is static in posture A. FIG. 4B is a diagram illustrating the subject who is static in posture B.

Subject 11 shown in FIG. 4A or FIG. 4B takes a static posture while sitting on chair 12 as in the example shown in FIG. 1A. Note that although a table, a PC, and so on that are not shown in FIG. 4A or FIG. 4B are actually present, only subject 11 and chair 12 are illustrated in the figures. The static posture of subject 11 shown in FIG. 4A is posture A which imposes a relatively large load on the shoulders while the static posture of subject 11 shown in FIG. 4B is posture B which imposes a relatively small load on the shoulders.

A fatigue level estimated for subject 11 who is static in such posture A or posture B is accumulated as shown in FIG. 5A and FIG. 5B. FIG. 5A is a first diagram for explaining the accumulation of the estimated fatigue level of the subject according to the embodiment. FIG. 5B is a second diagram for explaining the accumulation of the estimated fatigue level of the subject according to the embodiment.

As illustrated in FIG. 5A, when the posture of subject 11 is static while keeping posture A shown in FIG. 4A or posture B shown in FIG. 4B, the fatigue level of subject 11 is expressed using a linear function indicating, as a slope, an amount of load calculated based on the posture.

As described above, posture A is a posture with a larger amount of load compared to posture B. Accordingly, an amount of load imposed on a certain muscle of subject 11 (a muscle related to the movability of the shoulders in this case) by posture A (the slope of a straight line indicating posture A) is larger than an amount of load imposed by posture B (the slope of a straight line indicating posture B). When subject 11 is in posture A, the fatigue level is accumulated (cumulative) much more in a short period of time compared to the case where subject 11 is static in posture B.

As illustrated in FIG. 5B, when the posture of subject 11 changes from posture A shown in FIG. 4A to posture B shown in FIG. 4B, the fatigue level of subject 11 is expressed by a function in which a linear function indicating, as a slope, an amount of load calculated based on the posture and a linear function indicating, as a slope, an amount of the change in the posture are joined together.

Accordingly, while subject 11 is static in posture A, the fatigue level of a certain muscle of subject 11 is estimated as the accumulation (increase) of the fatigue level, using an increasing function indicating a positive slope corresponding to an amount of load imposed by posture A, and the accumulation (increase) turns toward recovery (decrease) at a change point where subject 11 started to change the posture. By using a decreasing function indicating a negative slope corresponding to an amount of the change in the posture, the fatigue level of subject 11 is recovered (decreased) for an amount indicated as a change width in the FIG. 5B in a time period, which is indicated as a period of change in the figure, during which the posture continues to change. After a change point at which the posture of subject 11 became static again in posture B, the fatigue level of subject 11 is estimated as the accumulation (increase) of the fatigue level, using an increasing function indicating a positive slope corresponding to an amount of load imposed by posture B.

In this way, the fatigue level of subject 11 where accumulation and recovery are reflected in accordance with keeping and changing of the posture of subject 11 is estimated in fatigue estimation system 200 according to the present embodiment.

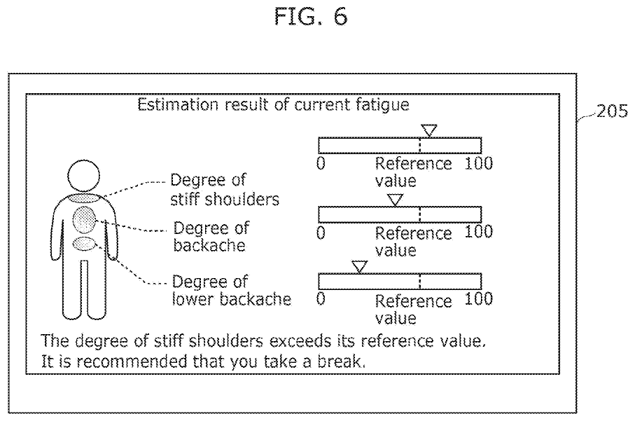

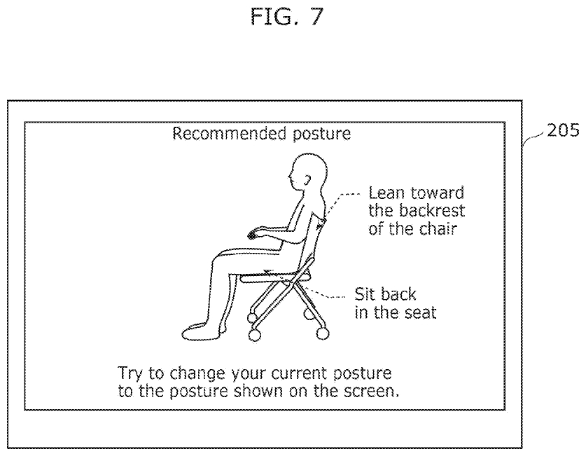

Next, an example of output by output unit 109 based on an estimated fatigue level will be described. FIG. 6 is a first diagram illustrating an example of a display of an estimation result according to the embodiment. FIG. 7 is a second diagram illustrating an example of a display of an estimation result according to the embodiment.

As illustrated in FIG. 6 and FIG. 7, fatigue estimation system 200 is capable of feeding back the estimation result of the fatigue level of subject 11 to subject 11 by displaying the estimation result using display device 205. Specifically, by visualizing the fatigue levels of subject 11, as shown in FIG. 6, it is possible for subject 11 to visually grasp how tired subject 11 is. In FIG. 6, a figure modeling subject 11 and the levels of fatigue at the shoulders, back, and lower back of subject 11 are displayed in an integrated manner by display device 205. In order to make it easier for subject 11 to see the fatigue levels at first sight, the fatigue level of the shoulders is indicated as “the degree of stiff shoulders”, the fatigue level of the back is indicated as “the degree of backache”, and the fatigue level of the lower back is indicated as “the degree of lower backache”.

The fatigue levels of three locations in subject 11 are all displayed in the display example in FIG. 6, and the fatigue levels of the three locations are estimated from images captured in one sequence of imaging. In other words, estimation device 100 estimates, from one posture of subject 11, a fatigue level regarding a muscle and/or a joint at each of the body parts including a first part (e.g., shoulders), a second part (e.g., a back), and a third part (e.g., a lower back) of subject 11. Accordingly, even though the posture of subject 11 is the same, the level of fatigue accumulated in the muscle and/or the joint is different among the body parts, and yet fatigue estimation system 200 is capable of estimating such different fatigue levels simultaneously and separately.

In the present embodiment, since an amount of load is calculated for each of muscles and/or joints of subject 11, as described with reference to FIG. 1C, it is possible to estimate the fatigue level of each of the muscles and/or joints as long as a processing resource is not limited. Accordingly, the number of body parts for which the fatigue levels are estimated from images captured in one sequence of imaging is not limited, and may be one, two, or four or more.

Estimation device 100 is capable of calculating an amount of load for each of the body parts and estimating, for a single posture of subject 11, the fatigue level of the first part (the degree of stiff shoulders described above) based on an amount of load calculated for the first part, the fatigue level of the second part (the degree of backache described above) based on an amount of load calculated for the second part, and the fatigue level of the third part (the degree of lower backache described above) based on an amount of load calculated for the third part.

In the example in FIG, 6, the degree of stiff shoulders is estimated from an amount of load imposed on trapezius, the degree of backache is estimated from an amount of load imposed on latissimus dorsi, and the degree of lower backache is estimated from an amount of load imposed on lumbar paraspinal muscles. A fatigue level may be estimated from an amount of load imposed on a single muscle and/or a single joint, but may be estimated from multiple amounts of loads imposed on muscles and/or joints. For example, the degree of stiff shoulders (i.e., the fatigue level of shoulders) may be estimated from the average value of amounts of loads on trapezius, levator scapulae, rhomboid, and deltoid. In the estimation of a fatigue level, a fatigue level that is close to reality may be estimated by weighting an amount of load imposed on a muscle and/or a joint which particularly significantly affects the fatigue level of a body part, instead of simply calculating the average value of all of amounts of loads imposed on muscles and/or joints related to the body part.

The fatigue levels thus estimated may be each indicated as a relative position on a reference indicator where the smallest value is 0 and the largest value is 100, as illustrated in FIG. 6. A reference value is set at a predetermined position on the reference indicator. Such a reference value is set at a relative position (or before or after the relative position, for instance) of the level of fatigue whose subjective symptoms such as a pain might appear in subject 11 in general and which is quantified in advance in an immunological examination, for instance. Accordingly, different values may be set for reference values in accordance with the fatigue levels of the body parts.

Furthermore, display device 205 may display a warning message to subject 11 as an estimation result when triggered by the estimated fatigue level of subject 11 reaching its reference value. The reference value here is an example of a first threshold. In FIG. 6, “The degree of stiff shoulders exceeds its reference value.” is indicated as an example of such a warning message, as displayed in the lower part of the screen of display device 205. Moreover, in association with such a warning message, display device 205 may display a specific way to deal with the situation, such as an advice “It is recommended that you take a break.” as indicated together with the warning message in the figure.

Alternatively, when triggered by the estimated fatigue level of subject 11 reaching its reference value, display device 205 may display a recommended posture that achieves a less amount of load on a body part of which the fatigue level has reached its reference value, compared to the currently estimated posture of subject 11. The reference value here is an example of a second threshold, and may be same as or different from the first threshold. Detailed advices such as “Lean toward the backrest of the chair.” and “Sit back in the seat.” may be indicated together with a figure that takes the displayed recommended posture.

Besides the above configuration for urging subject 11 to deal with an accumulated fatigue level, by displaying the estimation result of the fatigue level to subject 11, a configuration in which fatigue estimation system 200 actively recovers the fatigue level of subject 11 is also conceivable. Specifically, the fatigue level of subject 11 is recovered by recovery device 206 shown in FIG. 2 operating. Since the detailed configuration of recovery device 206 is as described above, description is omitted. Recovery device 206 operates when triggered by the estimated fatigue level of subject 11 reaching its reference value, changes a load imposed on at least one of a muscle or a joint of subject 11, and decreases the fatigue level of subject 11 by promoting the blood circulation of subject 11. The reference value here is an example of a third threshold and may be same as or different from either one of the first threshold and the second threshold.

### Advantageous Effects, etc.

As described above, fatigue estimation system 200 according to the present embodiment includes: an information output device (e.g., imaging device 201) that outputs information regarding the locations of the body parts of subject 11; and estimation device 100 that estimates the posture of subject 11 based on the information (e.g., images) output by the information output device, and estimates the fatigue level of subject 11 based on the estimated posture and the duration of the estimated posture.

For example, the information output device may be imaging device 201 that captures images of subject 11 and outputs the images as the information regarding the locations of the body parts of subject 11. Estimation device 100 may estimate the posture of subject 11 based on the images output by imaging device 201.

Such fatigue estimation system 200 is capable of estimating the fatigue level of subject 11, using images output by imaging device 201. In the estimation of the fatigue level of subject 11, the posture of subject 11 estimated from the output images is used. Specifically, the accumulation of fatigue caused by, for instance, an amount of load imposed on a muscle, an amount of load imposed on a joint, and a deteriorating blood flow of subject 11 which result from keeping of a same static posture are quantified as fatigue levels based on duration in which subject 11 is static in the static posture. Thus, with fatigue estimation system 200, it is possible to estimate the fatigue level of subject 11 in the static posture with higher accuracy and a less amount of load imposed on subject 11 since the fatigue level of subject 11 is calculated, based on images, in consideration of the duration in which the static posture continues.

For example, estimation device 100 may (i) calculate, using a musculo-skeletal model, an amount of load imposed on at least one of a muscle or a joint of subject 11 required for keeping the estimated posture, and (ii) estimate the fatigue level of subject 11 using an increasing function indicating the fatigue level with respect to the duration of the estimated posture. In the increasing function used for the estimation of the fatigue level, the fatigue level may increase per unit time as the calculated amount of load increases.

Accordingly, an amount of load regarding at least one of each muscle or each joint is calculated using a musculo-skeletal model. With an increasing function indicating, as a slope, the amount of load thus calculated, it is possible to readily estimate the fatigue level of subject 11. It is therefore possible to estimate the fatigue level of subject 11 with ease and higher accuracy.

For example, estimation device 100 may (i) calculate an amount of load of at least one of a muscle or a joint of each of two or more body parts of subject 11 including a first part and a second part among the body parts of subject 11, and (ii) estimate, for one posture of subject 11, at least a first fatigue level of the first part based on the amount of load calculated for the first part and a second fatigue level of the second part based on the amount of load calculated for the second part.

Accordingly, it is possible to calculate the fatigue level of each of two or more of the body parts of subject 11 in one sequence of imaging. Thus, there is no need to perform measurement for estimating the fatigue level for each of the body parts, and it is possible to promptly and almost simultaneously estimate the fatigue level for each of the body parts. Moreover, since a body part that is prone to get tired among all of the body parts of subject 11 can be easily identified based on the fatigue levels that have been estimated almost simultaneously, fatigue estimation system 200 is effective in providing a way to recover the fatigue level. It is therefore possible to promptly and effectively estimate the fatigue level of subject 11.

For example, when the posture changes, estimation device 100 may estimate the fatigue level using a decreasing function indicating the fatigue level with respect to time. In the decreasing function used for the estimation of the fatigue level, the fatigue level may decrease per unit time as the amount of the change in the posture increases.

Accordingly, with a change in the posture of subject 11, the recovery of the fatigue level, which owes to changing an amount of load imposed on at least one of a muscle or a joint of subject 11 and improving the blood circulation of subject 11, is reflected in the fatigue level to be estimated. It is therefore possible to estimate the fatigue level of subject 11 with higher accuracy.

For example, fatigue estimation system 200 may further include display device 205 that displays, to subject 11, a warning message as the result of the estimation of the fatigue level, where the display of the warning message is triggered by the fatigue level of subject 11, which is estimated by estimation device 100, reaching a first threshold.

Accordingly, subject 11, for instance, can know that the fatigue level of subject 11 has reached the first threshold, owing to a warning message displayed by display device 205. It is thus possible for subject 11 to inhibit the risk of experiencing a bad condition such as ill health, an injury, and an accident due to fatigue, by dealing with an accumulated fatigue level according to the displayed warning message. Thus, a bad condition caused by the fatigue of subject 11 is inhibited using a fatigue level estimated with higher accuracy.

For example, fatigue estimation system 200 may further include display device 205 that displays, to subject 11, a recommended posture that achieves a less amount of load compared to an amount of load imposed by the estimated posture, where the display of the recommended posture is triggered by the fatigue level of subject 11, which is estimated by estimation device 100, reaching a second threshold.

Accordingly, subject 11, for instance, can deal with a fatigue level that has reached the second threshold, owing to a recommended posture displayed by display device 205. Since the recovery of the fatigue level of subject 11 is expected by changing the current posture to the recommended posture, subject 11 can inhibit the accumulation of fatigue without being particularly aware of it. Thus, a bad condition caused by the fatigue of subject 11 is inhibited by using a fatigue level estimated with higher accuracy.

For example, fatigue estimation system 200 may further include recovery device 206 that lowers the fatigue level of subject 11 by promoting the blood circulation of subject 11, where the lowering of the fatigue level is triggered by the fatigue level of subject 11, which is estimated by estimation device 100, reaching a third threshold.

Accordingly, since the recovery of the fatigue level of subject 11 is expected owing to recovery device 206, subject 11 can inhibit the accumulation of fatigue without being particularly aware of it. Thus, a bad condition caused by the fatigue of subject 11 is inhibited by using a fatigue level estimated with higher accuracy.

For example, fatigue estimation system 200 may further include pressure sensor 203 that outputs a pressure distribution indicating a distribution of pressure given to the detection face of pressure sensor 203. Estimation device 100 may correct the estimated posture of subject 11 based on the pressure distribution output by pressure sensor 203, and calculate an amount of load required for keeping the corrected posture.

Accordingly, it is possible to use the pressure distribution output by pressure sensor 203 for the estimation of the posture of subject 11. Thus, the posture of subject 11 is estimated with higher accuracy owing to corrections made with the use of the pressure distribution. It is therefore possible to estimate the fatigue level of subject 11 with higher accuracy.

For example, fatigue estimation system 200 may further Include a receiving device that receives, as input, personal information including at least one of age, sex, height, weight, a muscle mass, a stress level, a proportion of fat in a body, or proficiency in performing exercise. When calculating an amount of load required for keeping the estimated posture, estimation device 100 may correct the amount of load based on the personal information received as the input by receiving device 204.

Accordingly, it is possible to use personal information received by receiving device 204 for the calculation of an amount of load. Thus, an amount of load imposed by a static posture is calculated with higher accuracy owing to corrections made with the use of the personal information. It is therefore possible to estimate the fatigue level of subject 11 with higher accuracy.

Estimation device 100 according to the present embodiment includes: first obtainer 101 that obtains information regarding the locations of the body parts of subject 11; posture estimator 105 that estimates the posture of subject 11 based on the information obtained by first obtainer 101; and fatigue estimator 108 that estimates the fatigue level of subject 11 based on the duration of the posture estimated by posture estimator 105.

Such estimation device 100 is capable of estimating the fatigue level of subject 11 using information such as obtained images. In the estimation of the fatigue level of subject 11, the posture of subject 11 estimated from, for instance, the obtained images is used. Specifically, the accumulation of fatigue caused by an amount of load imposed on a muscle, an amount of load imposed on a joint, and the deteriorating blood flow of subject 11 which result from keeping of a same static posture are quantified as fatigue levels based on duration in which subject 11 is static in the static posture. Since estimation device 100 calculates the fatigue level of subject 11 in consideration of the duration of the static posture, it is possible to estimate, with higher accuracy, the fatigue level of subject 11 in the static posture.

A fatigue estimation method according to the present embodiment includes: obtaining information regarding the locations of the body parts of subject 11 (e.g., step S103); estimating the posture of subject 11 based on the information obtained in the obtaining (step S106); and estimating the fatigue level of subject 11 based on the duration of the posture estimated in the estimating of the posture in step S106 (step S109).

Such a fatigue estimation method produces the same effects as those produced by estimation device 100 described above.

### OTHER EMBODIMENTS

Although an embodiment of the present disclosure is described above, the present disclosure is not limited to the embodiment.

For example, in the above embodiment, a process executed by a specific processing unit may be executed by another processing unit. An order of processes may be changed or processes may be executed in parallel.

The fatigue estimation system according to the present disclosure may be implemented by a plurality of devices each having one or more of the components of the fatigue estimation system or by a single device having all of the components. Likewise, the estimation device according to the present disclosure may be implemented by a plurality of devices each having one or more of the components of the estimation device or by a single device having all of the components. One or more of the functions of a component may be implemented as one or more functions of another component, or each of the functions may be distributed to any of components in any way. Any form with a configuration substantially including all of the functions achievable by the fatigue estimation system or the estimation device according to the present disclosure is included in the scope of the present disclosure.

In the above embodiment, the respective components may be implemented by executing software programs suited to the respective components. The respective components may be implemented by a program execution unit such as a CPU or a processor reading and executing a software program recorded on a recording medium such as a hard disk or semiconductor memory.

The respective components may be implemented by hardware. For example, the respective components may be circuits (or integrated circuits). These circuits may compose a single circuit as a whole or may be separate circuits. Moreover, these circuits may be general-purpose or dedicated circuits.

General or specific aspects of the present disclosure may be implemented using a system, a device, a method, an integrated circuit, a computer program, or a computer-readable recording medium such as a CD-ROM, or any combination of systems, devices, methods, integrated circuits, computer programs, and recording media.

Although the posture of a subject is estimated from images, using a rigid link model generated through image recognition, an amount of load is calculated based on the estimated posture of the subject and the fatigue level of the subject is estimated based on the amount of load and the duration of the estimated posture in the above embodiment, a method for estimating a fatigue level is not limited to such a method. Any existing method may be used as a method for estimating the posture of a subject from images, and any existing method may be used as a method for estimating an amount of load from the posture of a subject.

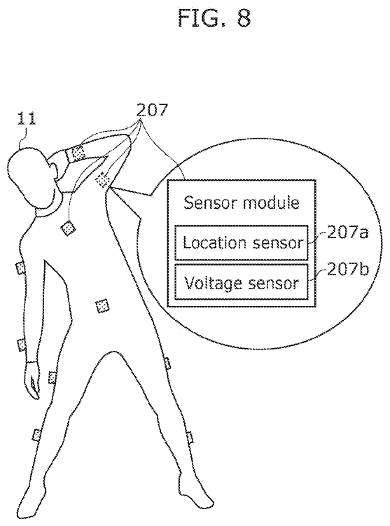

Moreover, it is also possible to implement the present disclosure by a configuration that uses a location sensor besides a configuration that uses an imaging device, as a method for estimating the posture of a subject. A specific example will be described with reference to FIG. 8. FIG. 8 is a diagram for explaining the estimation of a posture according to a variation of the embodiment. According to the present variation, the posture of subject 11 is estimated using sensor module 207 including location sensor 207a and voltage sensor 207b, as illustrated in FIG. 8. Although a plurality of sensor modules 207 are worn by subject 11, the number of sensor modules 207 to be worn by subject 11 is not particularly limited. Only one sensor module 207 may be worn by subject 11.

Moreover, how to wear sensor modules 207 is not particularly limited and any way may be allowed as long as the location of a predetermined body part of subject 11 can be measured. In FIG. 8, for example, sensor modules 207 are worn by subject 11 wearing clothing to which these sensor modules 207 are attached.

Sensor module 207 is a device worn by subject 11 on a predetermined body part and outputs information indicating the result of detection or measurement linked to the predetermined body part. Specifically, sensor module 207 includes location sensor 207a that outputs location information regarding the spatial position of the predetermined body part of subject 11, and voltage sensor 207b that outputs potential information indicating an electric potential at the predetermined body part of subject 11. Although sensor module 207 including both location sensor 207a and voltage sensor 207b is shown in FIG. 8, voltage sensor 207b is not essential if sensor module 207 includes location sensor 207a.

Location sensor 207a in such sensor module 207 is one example of an information output device that outputs information regarding the locations of the body parts of subject 11. Accordingly, the information to be output is location information and includes the relative or absolute location of a predetermined body part of subject 11. The information to be output may include, for example, potential information. The potential information is information including the value of an electric potential measured at a predetermined body part of subject 11. Hereinafter, the location information and the potential information will be described in detail together with location sensor 207a and voltage sensor 207b.

Location sensor 207a is a detector that detects the spatial relative or absolute location of a predetermined body part of subject 11 on which sensor module 207 is worn, and outputs information regarding the spatial location of the predetermined body part as the detection result. The information regarding the spatial location includes: information that can identify the location of a body part in a space, as described above; and information that can identify a change in the location of a body part resulting from body movement. Specifically, the information regarding the spatial location includes the locations of joints and skeletons in a space and information indicating a change in the locations.

Location sensor 207a is composed by combining various sensors such as an acceleration sensor, an angular velocity sensor, a geomagnetic sensor, and a ranging sensor. Since the location information output by location sensor 207a can be approximated to the spatial location of a predetermined body part of subject 11, it is possible to estimate the posture of subject 11 from the spatial location of a predetermined body part.

Voltage sensor 207b is a detector that measures an electric potential at a predetermined body part of subject 11 on which sensor module 207 is worn and that outputs information indicating the electric potential at the predetermined body part as the measurement result. Voltage sensor 207b is measuring equipment that includes electrodes and measures a potential generated between the electrodes using an electrometer. The potential information output by voltage sensor 207b indicates a potential generated at a predetermined body part of subject 11. Since the potential corresponds to, for instance, the active potential of a muscle at the predetermined body part, it is possible to enhance estimation accuracy in estimating the posture of subject 11 from, for instance, the active potential of the predetermined body part.

The fatigue estimation system according to the present variation estimates the fatigue level of subject 11, using the posture of subject 11 estimated as described above. Note that the processes following the estimation of the posture of subject 11 is the same as those described in the above embodiment, description is omitted.

As described above, in the fatigue estimation system according to the present variation: the information output device is location sensor 207a that is worn on a predetermined body part of subject 11 and that outputs location information regarding the spatial location of the predetermined body part as information regarding the locations of the body parts of subject 11; and estimation device 100 estimates the posture of subject 11 based on the location information output by location sensor 207a.

Accordingly, it is possible to estimate the fatigue level of subject 11, using the location information output by location sensor 207a. In the estimation of the fatigue level of subject 11, the posture of subject 11 estimated from the output information is used. Specifically, the accumulation of fatigue due to a same static posture being kept is quantified as a fatigue level based on duration in which subject 11 keeps the static posture. Thus, in the fatigue estimation system, since the fatigue level of subject 11 is calculated in consideration of the duration of a static posture and based on the results of the detection and measurement performed by sensor module 207, it is possible to estimate the fatigue level of subject 11 in a static posture with higher accuracy and a less amount of load imposed on subject 11.

Although the above embodiment has described that an increasing function and a decreasing function are linear functions, the functions are not limited to linear functions. The increasing function may be a curved function as long as it is a function in which a fatigue level increases with the elapse of time. The decreasing function may be a curved function as long as it is a function in which a fatigue level decreases with the elapse of time.

The above embodiment has described that the estimation device estimates the fatigue level of a subject, using the estimated values of an amount of load imposed on a muscle, an amount of load imposed on a joint, and the blood flow rate of the subject which are estimated from the posture of the subject. However, it is also possible to achieve the estimation of the fatigue level with higher accuracy by correcting the estimated values using values measured by a measurement device. Specifically, the estimation device obtains measured values which are based on the result of measuring the subject by the measurement device and correspond to the estimated values.

The detection device is, for example, an electromyograph, a muscle hardness tester, a manometer, or a near-infrared spectrometer, and is capable of obtaining, through measurement, measured values regarding an amount of load imposed on a muscle, an amount of load imposed on a joint, and a blood flow rate. For example, an electromyograph is capable of estimating, based on an electric potential measured through potential measurement, the movement of a muscle corresponding to the electric potential. In other words, a value resulting from the estimation of the muscle movement can be obtained as a measured value. Since the resulting value can be converted into an amount of load imposed on the muscle, it is possible to correct, using the measured value, the estimated value of the amount of load imposed on the muscle. The correction here is, for example, calculating an average value of the estimated value and the measured value, selecting one of the estimated value and the measured value, or substituting the estimated value into a correlation function between the estimated value and the measured value.

With a muscle hardness tester, it is possible to estimate the hardness of a muscle by a stress measured when pressure is given to the muscle. Since a value resulting from the estimation of the hardness of the muscle can be converted into an amount of load imposed on the muscle, the resulting value can be used for the correction of the estimated value of the amount of load imposed on the muscle, as is the case above.

With a manometer, it is possible to obtain, as a measured value, a result of examining what kind of pressure is imposed on a body part of a subject. A parameter of such pressure can be input to a musculo-skeletal model. By inputting an additional parameter such as pressure, the accuracy of the estimation using the musculo-skeletal model is enhanced and it is possible to correct, with higher accuracy, an estimated value estimated using the musculo-skeletal model.

With a near-infrared spectrometer, it is possible to obtain a measured value resulting from spectrographic measurement of the blood flow rate of a subject. As described in the above embodiment, when estimated values do not include a blood flow rate, the estimated values may be corrected by combining a blood flow rate measured by the near-infrared spectrometer. Even when estimated values include a blood flow rate, a measured blood flow rate may be used when the reliability of the estimated value of the blood flow rate is low.

By thus making corrections to obtain highly accurate estimated values using measured values corresponding to estimated values obtained from different aspects, it is possible to more accurately estimate the fatigue level of a subject.

A fatigue factor identification system that identifies the factors of fatigue of a subject may be configured with the use of the fatigue estimation system described in the above embodiment. With a conventional device or system that estimates a fatigue level as, for instance, “stiff shoulders” or “lower backache”, it has been difficult to identify how muscles and joints were used, which is a factor of such “stiff shoulders” or “lower backache” (i.e., a posture that is the factor). In view of this, it is possible to address the above problem by using the fatigue factor identification system according to the present disclosure.

In other words, in the fatigue factor identification system according to the present disclosure, a body part at which fatigue is prone to be accumulated (a body part of which an estimated amount that increases various fatigue is large) in a static posture taken by a subject is identified as a fatigue factor portion. The fatigue factor identification system may merely identify a fatigue factor portion in a single static posture taken by the subject or identify a fatigue factor posture of which an estimated amount in a fatigue factor portion is the largest among static postures taken by the subject. A recommended posture with which an identified fatigue factor posture is to be replaced may be presented to the subject or an operation of recovering the fatigue level of the subject with the use of the recovery device may be performed for a fatigue factor portion in a fatigue factor posture.

The fatigue factor identification system includes the fatigue estimation system described in the above embodiment and a storage device for storing information on an estimated fatigue level. Such a storage device is implemented using, for example, semiconductor memory. For instance, various storage units included in the fatigue estimation system may be used or a storage device communicably connected to the estimation device may be newly provided.

The present disclosure may be implemented as a fatigue estimation system or a fatigue estimation method to be executed by an estimation device. Moreover, the present disclosure may be implemented as a program for causing a computer to execute such a fatigue estimation method, or as a non-transitory computer-readable recording medium having such a program recorded thereon.

Various modifications to the embodiments which may be conceived by those skilled in the art, as well as embodiments resulting from arbitrary combinations of elements and functions from different embodiments are included within the scope of the present disclosure so long as they do not depart from the essence of the present disclosure.

### REFERENCE SIGNS LIST

11 subject

100 estimation device

101 first obtainer (obtainer)

105 posture estimator

108 fatigue estimator

200 fatigue estimation system

201 imaging device

203 pressure sensor

204 receiving device

205 display device

206 recovery device

207a location sensor

1. A fatigue estimation system comprising:
an information output device that outputs information regarding locations of body parts of a subject; and
an estimation device that estimates a posture of the subject based on the information output by the information output device, and estimates a fatigue level of the subject based on the posture estimated and a duration of the posture estimated, wherein
when the posture changes, the estimation device estimates the fatigue level using a decreasing function indicating the fatigue level with respect to a time period during which the posture continues to change, and
in the decreasing function used for the estimation of the fatigue level, the fatigue level decreases per unit time as an amount of the change in the posture increases.

2. The fatigue estimation system according to claim 1, wherein
the information output device is an imaging device that captures an image of the subject and outputs the image as the information, and
the estimation device estimates the posture of the subject based on the image output by the imaging device.

3. The fatigue estimation system according to claim 1, wherein
the information output device is a location sensor that is worn by the subject on a predetermined body part of the subject and that outputs, as the information, location information regarding a spatial location of the predetermined body part, and
the estimation device estimates the posture of the subject based on the location information output by the location sensor.

4. The fatigue estimation system according to claim 1, wherein
the estimation device (i) calculates, using a musculo-skeletal model or measured data, an estimated value of at least one of an amount of load imposed on a muscle, an amount of load imposed on a joint, or a blood flow rate of the subject which results from keeping of the posture estimated, and (ii) estimates the fatigue level using an increasing function indicating the fatigue level with respect to the duration, and
in the increasing function used for the estimation of the fatigue level, the fatigue level increases per unit time as the calculated estimated value increases.

5. The fatigue estimation system according to claim 4, wherein
the estimation device:
obtains a measured value that is based on a result of measuring the subject by a measurement device and corresponds to the estimated value; and
corrects the estimated value using the measured value obtained, and
in the increasing function used for the estimation of the fatigue level, the fatigue level increases per unit time as the corrected estimated value increases.

6. The fatigue estimation system according to claim 5, further comprising:
a receiving device that receives, as input, personal information including at least one of age, sex, height, weight, a muscle mass, a stress level, a proportion of fat in a body, or proficiency in performing exercise, wherein
when calculating the estimated value required for keeping the estimated posture, the estimation device corrects the estimated value based on the personal information received as the input by the receiving device.

7. The fatigue estimation system according to claim 4, wherein
the estimation device (i) calculates the estimated value for each of two or more body parts including a first part and a second part among the body parts of the subject, and (ii) estimates, for one posture of the subject, at least a first fatigue level of the first part based on the estimated value calculated for the first part and a second fatigue level of the second part based on the estimated value calculated for the second part.

8. The fatigue estimation system according to claim 4, further comprising:
a pressure sensor that outputs a pressure distribution indicating a distribution of pressure given to a detection face of the pressure sensor, wherein
the estimation device corrects the estimated posture of the subject based on the pressure distribution output by the pressure sensor, and calculates the estimated value required for keeping the corrected posture.

9. (canceled)

10. The fatigue estimation system according to claim 1, further comprising:
a display device that displays, to the subject, a warning message as a result of the estimation of the fatigue level, the display of the warning message being triggered by the fatigue level of the subject reaching a first threshold, the fatigue level being estimated by the estimation device.

11. The fatigue estimation system according to claim 1, further comprising:
a display device that displays, to the subject, a recommended posture that achieves at least one of a less amount of load imposed on a muscle, a less amount of load imposed on a joint, or a higher blood flow rate of the subject which results from keeping of the recommended posture, compared to an amount of load imposed on the muscle, an amount of load imposed on the joint, or a blood flow rate of the subject which results from keeping of the estimated posture, the display of the recommended posture being triggered by the fatigue level of the subject reaching a second threshold, the fatigue level being estimated by the estimation device.

12. The fatigue estimation system according to claim 1, further comprising:
a recovery device that lowers the fatigue level of the subject by promoting blood circulation of the subject, the lowering of the fatigue level being triggered by the fatigue level of the subject reaching a third threshold, the fatigue level being estimated by the estimation device.

13. An estimation device comprising:
an obtainer that obtains information regarding locations of body parts of a subject;
a posture estimator that estimates a posture of the subject based on the information obtained by the obtainer; and
a fatigue estimator that estimates a fatigue level of the subject based on a duration of the posture estimated by the posture estimator, wherein
when the posture changes, the fatigue estimator estimates the fatigue level using a decreasing function indicating the fatigue level with respect to a time period during which the posture continues to change, and
in the decreasing function used for the estimation of the fatigue level, the fatigue level decreases per unit time as an amount of the change in the posture increases.

14. A fatigue estimation method comprising:
obtaining information regarding locations of body parts of a subject;
estimating a posture of the subject based on the information obtained in the obtaining; and
estimating a fatigue level of the subject based on a duration of the posture estimated in the estimating of the posture, wherein
in the estimating of the fatigue level, when the posture changes, the fatigue level is estimated using a decreasing function indicating the fatigue level with respect to a time period during which the posture continues to change, and
in the decreasing function used for the estimation of the fatigue level, the fatigue level decreases per unit time as an amount of the change in the posture increases.
